(19) United States
(12) Patent Application Publication
(10) Pub. No.: US 2019/0296279 A1
(43) Pub. Date: Sep. 26, 2019

(54) MEDICAL DEVICE

(71) Applicant: NIHON KOHDEN CORPORATION, Tokorozawa (JP)

(72) Inventor: Norihito Konno, Tokyo (JP)

(73) Assignee: NIHON KOHDEN CORPORATION, Tokorozawa (JP)

(21) Appl. No.: 16/360,864

(22) Filed: Mar. 21, 2019

(30) Foreign Application Priority Data
Mar. 23, 2018 (JP) 2018-056686

(51) Int. Cl. H01M 2/10; A61B 5/00; H01M 2/04

(52) U.S. Cl. CPC H01M 2/1044; H01M 2/0465; A61B 5/0002

(57) ABSTRACT

To suppress size increase of a structure while providing a mechanism for preventing falling-off of a button battery in a medical device. A detection unit detects biological information of a subject. A housing defines an accommodating chamber for accommodating a button battery for supplying electric power to the detection unit. A cover is capable of closing the accommodating chamber. A locking member is supported by the housing and locks the cover to the housing so as to close the accommodating chamber. A space allowing displacement of the locking member for relating locking is formed by the housing and the cover.

### CROSS-REFERENCE TO RELATED APPLICATION(S)

This application claims priority to and the benefit under 35 U.S.C. § 119(a) of the earlier filing date of Japanese Application No. JP 2018-056686 filed Mar. 23, 2018 which is incorporated herein by reference, in its entirety, for any purpose.

### BACKGROUND OF THE INVENTION

### Field of the Invention

The present disclosure relates to a medical device operated by electric power supplied from a button battery.

### Description of Related Art

WO2015/170893 (Patent Literature 1) discloses this kind of medical device. The medical device includes a detection unit that detects biological information of a subject. Power supply to the detection unit is performed by a button battery. Therefore, the medical device can be carried by the subject.

As the above medical device has a manner of use in which the device is carried by the subject to detect biological information of the subject, it is highly required to suppress size increase of a structure and to prevent falling-off of the button battery.

### SUMMARY OF THE INVENTION

An object of the present disclosure is to provide a technique for suppressing size increase of the structure while providing a mechanism for preventing falling-off of the button battery in the medical device.

In order to achieve the above object, a medical device according to an embodiment of the present invention includes a detection unit detecting biological information of a subject, a housing defining an accommodating chamber for accommodating a button battery for supplying electric power to the detection unit, a cover capable of closing the accommodating chamber and a locking member supported by one of the housing and the cover and locking the cover to the housing so as to close the accommodating chamber, in which a space allowing displacement of the locking member for releasing locking is formed by the housing and the cover.

### BRIEF DESCRIPTION OF THE DRAWINGS

FIGS. 1A and 1B show an outer appearance of part of a medical device according to an embodiment;

FIGS. 2A and 2B show a structure of the above medical device;

FIG. 3 shows an enlarged structure of part of the above medical device;

FIGS. 4A and 4B show an enlarged structure of part of a medical device according to another example;

FIG. 5 shows an enlarged structure of part of a medical device further according to another example; and

FIGS. 6A to 6D show a structure of a medical device further according to another example.

### DESCRIPTION OF EMBODIMENTS

Examples of an embodiment will be explained below in detail with reference to the attached drawings. In respective drawings, scales are suitably changed for allowing respective components to be explained to have recognizable sizes.

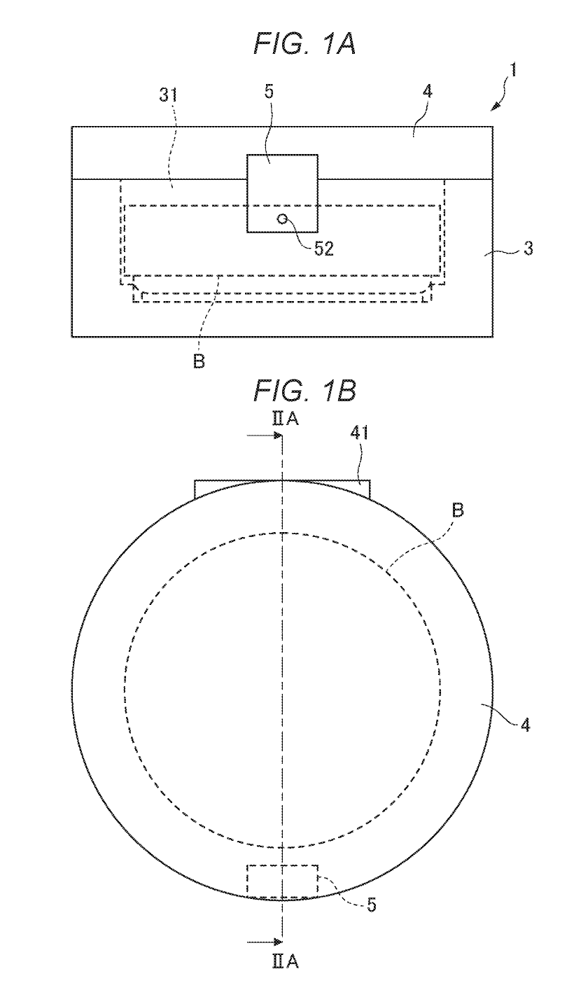

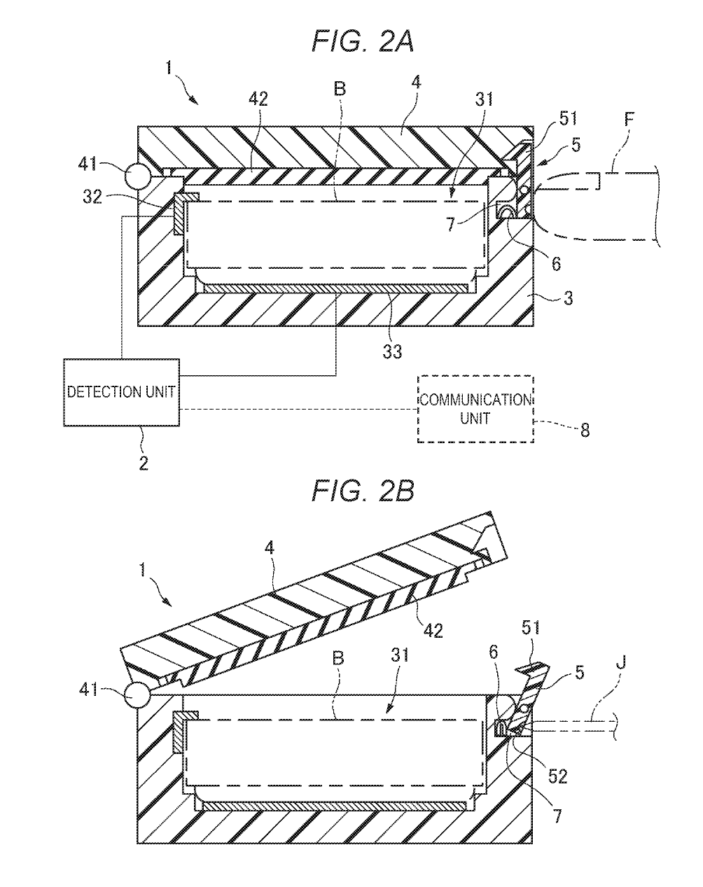

FIG. 1A shows an outer appearance seen from a side of part of a medical device 1 to an embodiment. FIG. 1B shows an outer appearance seen from the top of the medical device 1. FIG. 2A shows a structure of the medical device 1 while including a cross-sectional view taken along line IIA-IIA of FIG. 1B and seen from an arrow direction.

The medical device 1 is a device configured to be carried by a subject and to detect biological information of the subject. That is, the medical device 1 has a detection unit 2 for detecting biological information of the subject as shown in FIG. 2A.

As biological information, an electrocardiogram, brain waves, a body temperature, noninvasive arterial blood oxygen saturation (SpO2), pulse waves and so on can be cited as examples. The detection unit 2 has a configuration corresponding to biological information to be acquired. For example, when the electrocardiogram and brain waves are detected as biological information, the detection unit 2 may include at least one electrode attached to a body of the subject. When pulse waves and SpO2 are detected as biological information, the detection unit 2 may include a light emitting device and a light receiving device based on a pulse oximetry system. When the body temperature is detected as biological information, the detection unit 2 may include a temperature sensor attached to the body of the subject.

As shown in the above respective drawings, the medical device 1 includes a housing 3. The housing 3 defines an accommodating chamber 31 for accommodating a button battery B for supplying electric power to the detection unit 2.

In the accommodating chamber 31, a positive pole terminal 32 and a negative pole terminal 33 are arranged. The positive pole terminal 32 and the negative pole terminal 33 respectively contact a positive pole and a negative pole of the button battery B accommodated in the accommodating chamber 31. The positive pole terminal 32 and the negative pole terminal 33 are electrically connected to the detection unit 2. Accordingly, electric power is supplied from the button battery B to the detection unit 2.

As shown in the above respective drawings, the medical device 1 includes a cover 4. The cover 4 is configured to be able to close the accommodating chamber 31. In the example, the cover 4 is connected to the housing 3 through a hinge 41. As shown in FIG. 2B, the cover 4 can open the accommodating chamber 31 by pivoting about the hinge 41.

The cover 4 may be attached to and detached from the housing 3. In this case, the cover 4 closes the accommodating chamber 31 by being locked to the housing 3 through a locking claw or the like and opens the accommodating chamber 31 by being detached from the housing 3.

The cover 4 may be provided with a packing 42. The packing 42 is configured to block a gap generated between the housing 3 and the cover 4 when the cover 4 is arranged at a position where the cover 4 closes the accommodating chamber 31. Accordingly, it is possible to suppress intrusion of moisture or dust into the accommodating chamber 31.

The housing 3 and the cover 4 are respectively configured to have dimensions so as to be carried by a subject. The detection unit 2 may be integrally provided with the housing 3 as well as may be provided separately from the housing 3.

As shown in the above respective drawings, the medical device 1 includes a locking member 5. The locking member 5 is provided to lock the cover 4 to the housing 3 so as to close the accommodating chamber 31.

In the present example, the locking member 5 is supported by the housing 3. The locking member 5 includes an engaging portion 51 which can be engaged with the cover 4.

As shown in FIG. 2A, the medical device 1 includes an elastic member 6. The elastic member 6 is a spring such as a plate spring or a coil spring in the present example. The locking member 5 is biased toward an outer side in a radial direction of the button battery B by an elastic force of the elastic member 6. In a case where the cover 4 is positioned so as to close the accommodating chamber 31, the cover 4 is locked with the housing 3 by engaging the engaging portion 51 with the cover 4.

As shown in FIG. 2A, the housing 3 and the cover 4 define a space 7. As shown in FIG. 2B, the space 7 allows displacement of the locking member 5 in a direction resisting the elastic force due to the elastic member 6. When the locking member 5 is displaced to that direction by application of a suitable pressing force, engagement between the engaging member 51 and the cover 4 is released. Accordingly, the cover 4 may be displaced so as to open the accommodating chamber 31.

According to the structure, the locking member 5 for locking the cover 4 to the housing 3 accommodating the button battery B is provided by using the space 7 defined by the housing 3 and the cover 4. Therefore, size increase of the structure can be suppressed while providing a mechanism for preventing falling-off of the button battery B in the medical device 1.

A locking force of the locking member 5 is set to be higher than a force which can be applied from a user's finger to the direction resisting the elastic force of the elastic member 6 and to be lower than a force which can be applied from a prescribed jig to that direction. Such setting is made by determining at least one of a position, a shape and dimensions of the locking member 5 based on relation with respect to the elastic force of the elastic member 6.

For example, as shown in FIG. 1A, a small recess 52 may be formed on an outer face of the locking member 5. When the recess 52 is pressed by a tip end of a prescribed jig J as shown in FIG. 2B, a local force exceeding the elastic force of the elastic member 6 can be applied to the locking member 5. Accordingly, the locking member S is displaced within the space 7 in the direction resisting the elastic force of the elastic member 6, and engagement with respect to the cover 4 is released. The prescribed jig J may be a dedicated jig or may be a pen point of a ballpoint pen or the like.

On the other hand, it is difficult or incapable to apply the local force exceeding the elastic force of the elastic member 6 even when the locking member 5 is pressed by a user's finger F of as shown in FIG. 2A. Therefore, the locking member 5 is not displaced in the direction resisting the elastic force of the elastic member 6 and the locked state with the cover 4 is maintained.

According to the above structure, probability of occurrence of a situation in which the locking member 5 is released due to an accidental physical contact that may occur while carrying the medical device 1 can be reduced. Therefore, probability of occurrence of a situation in which the button battery B falls off during operation of the medical device 1 can be reduced.

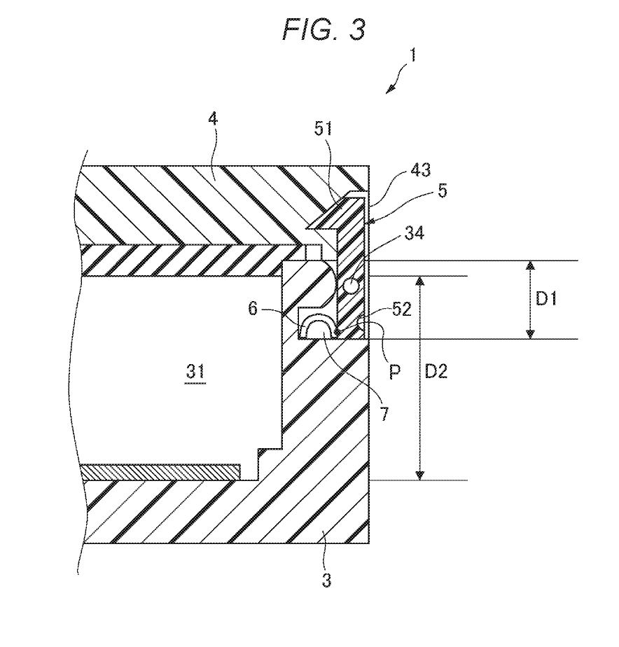

In the present example, as shown in FIG. 3, a dimension D1 of the space 7 extending along a thickness direction of the button battery B is smaller than a dimension D2 of the accommodating chamber 31 in the same direction.

Also according to the above structure, the local force exceeding the elastic force of the elastic member 6 is hardly applied to the locking member 5 and the probability of occurrence of the situation in which the locking member 5 is released due to an accidental physical contact that may occur while carrying the medical device 1 can be reduced. Therefore, the probability of occurrence of the situation in which the button battery B falls off during operation of the medical device 1 can be reduced.

In the present example, as shown in FIG. 3, the locking member 5 can pivot about a fulcrum 34 provided in the housing 3. The fulcrum 34 is arranged at a position closer to an application point P of the elastic force in the elastic member 6 than to the engaging portion 51.

Also according to the above structure, the local force exceeding the elastic force of the elastic member 6 is hardly applied to the locking member 5 and the probability of occurrence of the situation in which the locking member 5 is released due to an accidental physical contact that may occur while carrying the medical device 1 can be reduced. Therefore, the probability of occurrence of the situation in which the button battery B falls off during operation of the medical device 1 can be reduced.

In the present example, as shown in FIG. 3, the engaging portion 51 of the locking member 5 does not protrude from an outer edge 43 of the cover 4 to the outer side in the radial direction of the button battery B.

According to the above structure, increase in dimensions of the medical device 1 in the radial direction of the button battery B can be suppressed. It is also possible to inhibit a user's action to release locking of the locking member 5 by hooking a nail tip or the like on the engaging portion 51. Furthermore, probability of occurrence of a situation in which locking is accidentally released by some kind of substance being hooked on the engaging portion 51 while carrying the medical device 1 can be reduced. Therefore, the probability of occurrence of the situation in which the button battery B falls off during operation can be reduced while suppressing size increase of the medical device 1.

As shown in FIG. 2A, the medical device 1 may include a communication unit 8. The communication unit 8 may be configured to wirelessly transmit a signal corresponding to biological information detected by the detection unit 2 to an external apparatus. For example, the communication unit 8 may include communication interfaces conforming to standards such as RFID (Radio Frequency Identification), NFC (Near Field Communication), Bluetooth (trademark) and Wi-Fi.

According to the above configuration, the medical device 1 can be configured as a wireless sensor and a degree of freedom of a subject can be improved.

In a case where the signal output through the communication unit 8 is not performed, the medical device 1 may be provided with a not-shown storage unit. The storage unit may be configured by a storage such as a semiconductor memory or a hard disc drive. Biological information detected by the detection unit 2 is stored in the storage unit as data, which can be read out according to need.

The above embodiment is just an exemplification for making the present disclosure easy to understand. The configurations relating to the above embodiment can be suitably altered and improved within a scope not departing from the gist of the present invention.

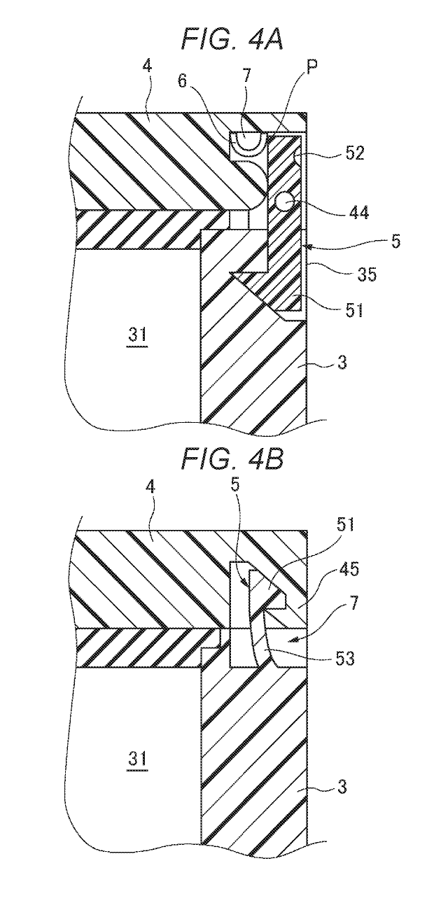

In the above embodiment, the locking member 5 is supported by the housing 3 and includes the engaging portion 51 which can be engaged with the cover 4. However, the locking member 5 may be supported by the cover 4 as shown in FIG. 4A. In this case, the engaging portion 51 is engaged with the housing 3. The engaging portion 51 may be configured so as not to protrude from an outer edge 35 of the housing 3 to the outer side in the radial direction of the button battery B. The locking member 5 can be pivot about a fulcrum 44 provided in the cover 4. The fulcrum 44 may be arranged at a position closer to the application point P of the elastic force in the elastic member 6 than to the engaging portion 51.

It is also preferable that the locking members 5 are provided at both the housing 3 and the cover 4. In this case, one locking member 5 provided in the housing 3 is engaged with the cover 4 and the other locking member 5 provided in the cover 4 is engaged with the housing 3.

In the above embodiment, the elastic member 6 is the spring. However, the elastic member 6 may be replaced with a rubber pad or the like as long as the locking member 5 can be biased toward the outer side in the radial direction of the button battery B.

In the above embodiment, the locking member 5 is biased toward the outer side in the radial direction of the button battery B by the elastic member 6. However, it is also preferable that the locking member 5 itself is configured to generate the elastic force. FIG. 4B shows such another example.

In the present example, when the cover 4 is displaced to a position where the cover 4 closes the accommodating chamber 31, the locking member 5 bends toward an inner side in the radial direction of the button battery B, and the engaging portion 51 climbs over an engaging projection 45 provided in the cover 4 to establish an engaged state. In this case, the locking member 5 is biased toward the outer side in the radial direction of the button battery B due to an elastic restoration force of itself to maintain the engaged state with respect to the cover 4. When releasing the engaged state, a base end portion 53 of the locking member 5 is pressed by a prescribed jig. Accordingly, the locking member 5 bends toward the inner side in the radial direction of the button battery B inside the space 7 and the engagement between the locking portion 51 and the engaging projection 45 is released.

Also in the example shown in FIG. 4B, the relation between the housing 3 supporting the locking member 5 and the cover 4 with which the locking member 5 is engaged can be reversed as in the example shown in FIG. 4A.

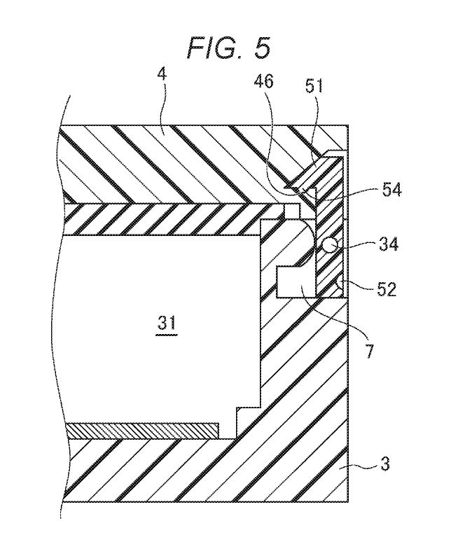

In the above embodiment, the elastic member 6 biases the locking member 5 toward the outer side in the radial direction of the button battery B. According to the above structure, the state where the cover 4 is locked to the housing 3 can be easily maintained. However, it is not always necessary that the medical device 1 includes the elastic member 6 if a desired locking force can be obtained. FIG. 5 shows such another example.

In the present example, a convex portion 54 is formed in the engaging portion 51 of the locking member 5. On the other hand, a concave portion 46 is formed in the cover 4. When the convex portion 54 is engaged with the concave portion 46, the cover 4 is locked to the housing 3. The space 7 defined by the housing 3 and the cover 4 allows displacement of the locking member 5 for releasing locking. When a lower part of the locking member 5 is displaced to the inner side in the radial direction of the button battery B by application of a suitable pressing force, engagement between the convex portion 54 and the concave portion 46 is released. Accordingly, the cover 4 may be displaced so as to open the accommodating chamber 31.

The locking force by the convex portion 54 and the concave portion 46 is set to be higher than a force which can be applied from a user's finger to the locking member 5 for releasing locking and to be lower than a force which can be applied from a prescribed jig to that direction for releasing locking.

Also in the example shown in FIG. 5, the relation between the housing 3 supporting the locking member 5 and the cover 4 with which the locking member 5 is engaged can be reversed as in the example shown in FIG. 4A.

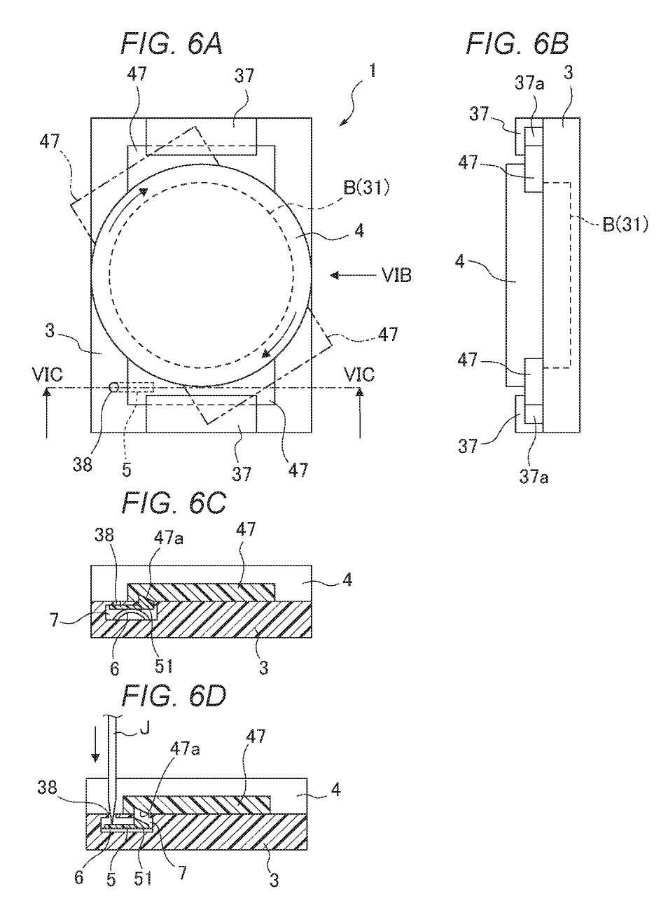

The biasing direction of the locking member 5 is not limited to the radial direction of the button battery B. FIG. 6A shows an outer appearance of the medical device 1 according to another example. FIG. 6B shows an outer appearance of the medical device 1 seen from a direction of an arrow VIB in FIG. 6A. FIG. 6C shows a cross section of the medical device 1 taken along line VIC-VIC in FIG. 6A and seen from an arrow direction.

In the present example, the cover 4 can pivot with respect to the housing 3 in a circumferential direction of the button battery B. The housing 3 includes a pair of pressing portions 37. The cover 4 includes a pair of flange portions 47. In lower parts of respective pressing portions 37, spaces 37a which can receive corresponding flange portions 47 are defined. The respective flange portions 47 enter corresponding spaces 37a by rotating the cover 4 to an arrow direction from a state shown by double chain lines in FIG. 6A.

In the present example, the locking member 5 is biased toward an outer side of the accommodating chamber 31 in the thickness direction of the button battery B by the elastic member 6 housed in the space 7 formed in the housing 3. The locking member 5 is allowed to be displaced in an upper and lower direction in the space 7, The locking member 5 includes the engaging portion 51. The engaging portion 51 has an inclined portion.

The flange portion 47 entering the space 37a abuts on the inclined portion of the engaging portion 51 and push down the locking member 5 to the lower part while resisting the biasing force of the elastic member 6. Accordingly, the flange portion 47 can move above the locking member 5 pressed into the space 7. A concave portion 47a is formed on a lower surface of the flange portion 47, When the flange portion 47 reaches a predetermined position in the space 37a, the engaging portion 51 faces the concave portion 47a. At this time, displacement of the locking member 5 to an upper direction is allowed due to the biasing force of the elastic member 6. When the locking portion 51 is engaged with the convex portion 47a, the locking member 5 is engaged with the cover 4. As a result, not only displacement of the cover 4 to the upper direction is restricted by the pair of pressing portions 37 but also displacement of the button battery B in the circumferential direction is restricted.

A hole 38 is formed in the housing 3. The hole 38 communicates with the space 7. When the locking member 5 housed in the space 7 is pressed by a tip end of a prescribed jig J as shown in FIG. 6D, a local force exceeding the elastic force of the elastic member 6 can be applied to the locking member 5. Accordingly, the locking member 5 is displaced downward in the space 7 while resisting the elastic force of the elastic member 6, and the engagement with the cover 4 is released. As a result, the cover 4 can be displaced in the circumferential direction of the button battery B, and engagement with respect to the housing 3 can be released.

In the present example, dimensions of the hole 38 are fixed so as not to be accessed to the locking member 5 by a user's finger.

In the present example, a dimension of the space 7 extending along the thickness direction of the button battery B is smaller than a dimension of the accommodating chamber 31 in the same direction.

The medical device 1 according to the present example may include the above-described communication unit 8, though not shown.

In order to achieve the above object, a medical device according to an embodiment of the present invention includes a detection unit detecting biological information of a subject, a housing defining an accommodating chamber for accommodating a button battery for supplying electric power to the detection unit, a cover capable of closing the accommodating chamber and a locking member supported by one of the housing and the cover and locking the cover to the housing so as to close the accommodating chamber, in which a space allowing displacement of the locking member for releasing locking is formed by the housing and the cover.

According to the above structure, the cover is locked to the housing by the locking member in a state where the accommodating chamber of the housing accommodating the button battery is closed by the cover. On the other hand, displacement of the locking member for releasing locking of the cover at the time of replacing the button battery or other cases is allowed by the space defined by the housing and the cover. Therefore, it is possible to suppress size increase of the structure while providing the mechanism for preventing falling-off of the button battery in the medical device.

What is claimed is:

1. A medical device comprising:
a detection unit configured to detect biological information of a subject;
a housing defining an accommodating chamber for accommodating a button battery for supplying electric power to the detection unit;
a cover capable of closing the accommodating chamber; and
a locking member supported by one of the housing and the cover and configured to lock the cover to the housing so as to close the accommodating chamber,
wherein a space allowing displacement of the locking member for releasing locking is formed by the housing and the cover.

2. The medical device according to claim 1,
wherein the locking member is biased toward an outer side in a radial direction of the button battery by an elastic force, and
a space allowing displacement of the locking member in a direction resisting the elastic force is formed by the housing and the cover.

3. The medical device according to claim 1,
wherein a locking force of the locking member is higher than a force capable of being applied from a user's finger to the locking member for releasing locking and lower than a force capable of being applied from a prescribed jig to the locking member for releasing locking.

4. The medical device according to claim 1,
wherein a dimension of the space extending along a thickness direction of the button battery is smaller than a dimension of the accommodating chamber in that direction.

5. The medical device according to claim 2,
wherein the locking member is capable of pivoting about a fulcrum provided at the one of the housing and the cover, and
the locking member includes an engaging portion engaged with another one of the housing and the cover, and
the fulcrum is arranged at a position closer to an application point of the elastic force than to the engaging portion.

6. The medical device according to claim 1,
wherein the locking member includes an engaging portion engaged with another of the housing and the cover, and
the engaging portion does not protrude from an outer edge of the other of the housing and the cover to an outer side in a radial direction of the button battery.

7. The medical device according to claim 1, further comprising:
a communication unit configured to wirelessly transmit a signal corresponding to the biological information.

8. A medical device comprising:
a detection unit configured to detect biological information of a subject;
a housing defining an accommodating chamber for accommodating a button battery for supplying electric power to the detection unit;
a cover closing the accommodating chamber by being locked to the housing and releasing locking with respect to the housing by being rotated in a circumferential direction of the button battery; and
a locking member supported by the housing and preventing rotation of the cover by being engaged with the cover,
wherein a space allowing displacement of the locking member for releasing locking with respect to the cover is formed in the housing.

9. The medical device according to claim 8,
wherein the housing includes a member configured to restrict displacement of the cover to a thickness direction of the button battery.

10. The medical device according to claim 8,
wherein the locking member is biased toward an outer side of the accommodating chamber in the thickness direction of the button battery by an elastic force, and
a space allowing displacement of the locking member in a direction resisting the elastic force is formed in the housing.

11. The medical device according to claim 8,
wherein a locking force of the locking member is higher than a force capable of being applied from a user's finger to the locking member for releasing locking and lower than a force capable of being applied from a prescribed jig to the locking member for releasing locking.

12. The medical device according to claim 8,
wherein a dimension of the space extending along a thickness direction of the button battery is smaller than a dimension of the accommodating chamber in that direction.

13. The medical device according to claim 8, further comprising:
a communication unit configured to wirelessly transmit a signal corresponding to the biological information.
